(19) United States
(12) Patent Application Publication
(10) Pub. No.: US 2003/0171545 A1
(43) Pub. Date: Sep. 11, 2003

(54) Novel Protein

(76) Inventors: Diane Joan Cousens, Hertfordshire (GB); Diane Michele Ignar, Durham, NC (US); Philippe Sanseau, Stevenage (GB); Filippo Volpe, Stevenage (GB)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/130,845

(22) PCT Filed: Dec. 1, 2000
(86) PCT No.: PCT/GB00/04606

(30) Foreign Application Priority Data
Dec. 2, 1999 (GB) 9928539.7
May 24, 2000 (GB) 0012699.5

(51) Int. Cl. C07K 14/705; G01N 33/53; C12P 21/02; A61K 38/17

(52) U.S. Cl. 530/350; 435/7.1; 514/12

(57) ABSTRACT

A method for in a the identification of a compound which modulates leukotriene B4 like (LTRGW1) receptor activity comprises contacting an LTRGW1 polypeptide comprising) The amino acid sequence of SEQ ID #2, or ii) A variant of (i) which is capable of binding leukotrienes: or iii) A fragment of (i) or (ii) which is capable of binding leukotrienes. Additionally. LTRGW1 has been determined to enhance the response of the leukotriene B4 Receptor (BLTR) to LTB4. Hence LTRGW1 may be used in methods to indentify compounds which modulate BLTR receptor activity.

### FIELD OF THE INVENTION

The present invention relates to methods of screening for modulators of leukotriene-B₄ receptor-like polypeptides, and use of leukotriene-B₄ receptor like polypeptides in methods of screening modulators of the leukotriene B₄ receptor, BLTR.

### BACKGROUND OF THE INVENTION

Phospholipid undergoes metabolic degradation to form arachidonic acid which may be further metabolised to produce leukotrienes (LT) such as LTB₄, LTD₄, LTE₄, LTC₄ and LTF₄. There are two main classes of leukotriene receptor, the cysteinyl receptors and leukotriene-B₄ (BLT) receptors. One leukotriene-B₄ receptor, BLTR, has been cloned and pharmacologically characterised (Yokomitso et al. (1997) Nature 387, 620-624.) Analysis of the amino acid sequence of BLTR suggests that it belongs the G-protein coupled receptor superfamily, characterised by seven predicted transmembrane domains.

LTB₄ is a chemoattractant for neutrophils and primed eosinophils. LTB₄ enhances neutrophil-endothelial interactions and activates neutrophils leading to degranulation and release of mediators, enzymes and superoxides. LTB₄ is also able to bind and activate a nuclear transcription factor (PPAR_{α}). This activation results in the transcription of genes that are responsible for the termination of the immune response. In addition, the cloned LTB₄ receptor, BLTR, has been reported to mediate HIV-1 entry in CD4 positive T cells.

Diseases such as asthma are associated with deregulation of the host immune response. Hyper-responsiveness in asthmatic patients is characterised by eosinophilia, oedema and mucus production in the lung. Leukotriene receptor antagonists such as zafirlukast, pranlukast and montelukast are currently used to control the inflammatory response in asthmatic patients. Neutrophilic inflammation is a symptom of chronic obstructive pulmonary disease (COPD) and it is likely that LTB₄ receptor antagonists may also have clinical benefit in COPD patients.

### SUMMARY OF THE INVENTION

A leukotriene-B₄ receptor-like polypeptide (LTRGW1) is now provided which the inventors have shown to enhance the activity of BLTR in response to LTB₄ stimulation, as well as acting as a leukotriene receptor in its own right. Novel assays are provided which utilise the interaction between LTRGW1 and BLTR to identify and develop novel pharmaceutical agents, including agonists and antagonists of the BLTR leukotriene-B₄ receptor. LTRGW1 may also be utilised as a screening target for the identification and development of novel pharmaceutical agents, including agonists and antagonists of the receptor. LTRGW1 may further be utilised in a screen to discover modulators of the interaction between BLTR and LBT4. Also provided is a method of enhancing BLTR response to LBT4 comprising contacting a polypeptide of SEQ ID #2 or a variant thereof with BLTR. The present invention also provides a heterodimer comprising a polypeptide according to SEQ ID #2 and a polypeptide according to SEQ ID #8.

Accordingly, the present invention provides a method for the identification of a compound which modulates leukotriene B₄ like receptor (LTRGW1) activity, which method comprises contacting an LTRGW1 polypeptide comprising

(i) the amino acid sequence of SEQ ID NO: 2; or

(ii) a variant of (i) which is capable of binding leukotrienes; or

(iii) a fragment of (i) or (ii) which is capable of binding leukotrienes.

The invention also provides:

the use of an LTRGW1 polypeptide as herein defined to enhance BLTR responses to leukotrienes.

An isolated heterodimer comprising a LTRGW1 polypeptide as herein defined, and a BLTR polypeptide as herein defined.

A method for increasing the responsiveness of a screen for identification of a substance that modulates the activity of the leukotriene B₄ receptor (BLTR) comprising the addition to said screen of an LTRGW1 polypeptide as herein defined.

a method for identification of a substance that modulates BLTR activity, which method comprises contacting a polypeptide of the invention and a BLTR polypeptide comprising

(i) the amino acid sequence of SEQ ID NO: 8, or

(ii) A variant of (i) which is capable of binding LTB4; or

(iii) A fragment of (i) or (ii), which is capable of binding LTB4 with a test substance and monitoring for LTB₄ binding to the said polypeptides;

a method for identification of a substance that modulates leukotriene-B₄ receptor activity, which method comprises contacting an LTRGW1 polypeptide as herein defined and a BLTR polypeptide as herein defined with LTB₄ in the presence of a test substance and monitoring for leukotriene-B₄ receptor activity;

a method for identification of a substance that modulates leukotriene-B₄ receptor activity, which method comprises:

(i) providing

(a) an LTRGW1 polypeptide as herein defined;

(b) a BLTR polypeptide as herein defined; and

(c) a test substance

under conditions that would permit the interaction of (a) and (b) in the absence of (c);

(ii) monitoring the interaction between (a) and (b); and

(iii) determining whether (c) modulates the interaction between (a) and (b) and thereby determining whether the test substance is a modulator of leukotriene-B₄ receptor activity;

a test kit suitable for identification of a substance that modulates leukotriene-B₄ receptor activity, which kit comprises:

(a) an LTRGW1 polypeptide as herein defined which is capable of potentiating the activity of BLTR in response to LTB₄; and

(b) a BLTR polypeptide as herein defined.

a substance identified by one of the methods referred to above which stimulates or modulates leukotriene-B₄ receptor activity;

a method of treating a subject having a disorder that is responsive to LTRGW1 or BLTR receptor modulation, or modulation of the interaction between LTRGW1 and BLTR, which method comprises administering to said patient an effective amount of a substance of the invention; and

use of a substance of the invention in the manufacture of a medicament for the treatment or prophylaxis of a disorder that is responsive to modulation of LTRGW1 or BLTR receptor activity or modulation of the interaction between LTRGW1 and BLTR;

Preferably the disorder is selected from acute and chronic inflammatory diseases, asthma, chronic obstructive pulmonary disease (COPD), allergic rhinitis, hayfever, immune deficiency disorder, AIDS, rheumatoid arthritis, multiple sclerosis, leukemia, myesthenia gravis, graves disease, systemic lupus erythematosus, inflammatory bowel disease, encephalomyelitis, psoriasis, atopic dermatitis, septic shock, stroke, ischaemia reperfusion injury or cardiovascular disease. More preferred is when the disorder is an acute or chronic inflammatory diseases, asthma, chronic obstructive pulmonary disease (COPD) or psoriasis. Particularly preferred is when the disorder is asthma.

The invention further provides

A method of treating a patient with a respiratory disorder in particular asthma or chronic obstructive pulmonary disease (COPD), said method comprising the administration of a therapeutically effective amount of an antagonist of LTRGW1

A method of treating a respiratory disorder in particular asthma or chronic obstructive pulmonary disease (COPD), said method comprising the administration to a patient of a therapeutically effective amount of a substance that downregulates the interaction between LTRGW1 and BLTR, hence decreasing the response of BLTR to its ligand.

Use of a therapeutically effective amount of an antagonist of LTRGW1 in the manufacture of a medicament for the treatment or prophylaxis of respiratory diseases, in particular asthma or chronic obstructive pulmonary disease (COPD)

Use of an effective amount of a substance that downregulates the interaction between LTRGW1 and BLTR in the manufacture of a medicament for the treatment or prophylaxis of respiratory disorders, in particular asthma or chronic obstructive pulmonary disease (COPD)

### BRIEF DESCRIPTION OF THE FIGURES

FIG. 1 shows the dose dependent enhancement of BLTR activity by LTRGW1.

FIG. 2 shows the enhancement of LTB₄ binding to cells co-expresesing BLTR and LTRGW1.

FIG. 3 shows the tissue distribution of LTRGW1 mRNA.

FIG. 4 shows the tissue distribution of BLTR mRNA.

FIG. 5 shows the presence of BLTR and LTRGW1 transcripts in three different tissues.

FIG. 6 shows that BLTR and LTRGW1 co-immunoprecipitate.

### BRIEF DESCRIPTION OF THE SEQUENCES

SEQ ID No 1 shows the DNA and amino acid sequences of human LTRGW1.

SEQ ID No 2 is the amino acid sequence alone of LTRGW1.

SEQ ID NO: 3 shows the DNA and amino acid sequences of human LTRGW1-32.

SEQ ID NO: 4 is the amino acid sequence alone of LTRGW1-32.

SEQ ID NO: 5 shows the DNA and amino acid sequences of the short splice variant of human BLTR.

SEQ ID NO: 6 is the amino acid sequence alone of the short variant of BLTR.

SEQ ID NO: 7 shows the DNA and amino acid sequences of the long splice variant of human BLTR.

SEQ ID NO: 8 is the amino acid sequence alone of long variant of BLTR.

### DETAILED DESCRIPTION OF THE INVENTION

Throughout the present specification and the accompanying claims the words “comprise” and “include” and variations such as “comprises”, “comprising”, “includes” and “including” are to be interpreted inclusively. That is, these words are intended to convey the possible inclusion of other elements or integers not specifically recited, where the context allows.

The present invention relates to methods using a human leukotriene-B₄ receptor-like polypeptide, referred to herein as LTRGW1. LTRGW1 is also sometimes referred to as BLTR2. Sequence information for LTRGW1 is provided in SEQ ID NO: 1 (nucleotide and amino acid) and in SEQ ID NO: 2. Sequence information for a preferred fragment of LTRGW1 is provided in SEQ ID NO: 3 (nucleotide and amino acid) and in SEQ ID NO: 4.

The terms “LTRGW1 polypeptide”, “LTRGW1 receptor” and “LTRGW1” as used throughout the specification refer to a polypeptide comprising:

(i) the amino acid sequence of SEQ ID NO: 2; or

(ii) a variant of (i) which is capable of binding leukotrienes; or

(iii) a fragment of (i) or (ii) which is capable of binding leukotrienes.

Preferably, said variant or fragment is capable of binding LTB₄, 12-epi-LTB₄, LTB₃, LTB₅, LTD4, LTE₄, LTC₄, or LTF₄, particularly preferred is when said variant or fragment is capable of binding LTB₄.

The polypeptides are provided in isolated form. The term “isolated” is intended to convey that the polypeptide is not in its native state, insofar as it has been purified at least to some extent or has been synthetically produced, for example by recombinant methods. The term “isolated” therefore includes the possibility of the polypeptide being in combination with other biological or non-biological material, such as cells, suspensions of cells or cell fragments, proteins, peptides, expression vectors, organic or inorganic solvents, or other materials where appropriate, but excludes the situation where the polypeptide is in a state as found in nature.

AN LTRGW1 polypeptide may also be in a substantially purified form, in which case it will generally comprise the polypeptide in a preparation in which more than 50%, e.g. more than 80%, 90%, 95% or 97%, 98%, 99%, by weight of the polypeptide in the preparation is a polypeptide of the invention. Routine methods can be employed to purify and/or synthesise the proteins according to the invention. Such methods are well understood by persons skilled in the art, and include techniques such as those disclosed in Sambrook et al, Molecular Cloning: a Laboratory Manual, 2^{nd} Edition, CSH Laboratory Press (1989), the disclosure of which is included herein in its entirety by way of reference.

The term “variant” in relation to LTRGW1 refers to a polypeptide which has the same essential character or basic biological functionality as LTRGW1. It is preferred that fragments of LTRGW1 and/or fragments of a variant of LTRGW1 also possess the same essential character or basic biological functionality as LTRGW1. Any such variants or fragments are herein included within the defintion of LTRGW1 polypeptides.

In one aspect, the essential character of LTRGW1 can be defined as follows: LTRGW1 is a leukotriene-B₄ receptor-like polypeptide which interacts with BLTR. In this aspect, a polypeptide having the same essential character as LTRGW1 typically potentiates the activity of LTB₄ at BLTR. A polypeptide having the same essential character as LTRGW1 may be identified by co-expressing the polypeptide with BLTR and monitoring the effect on BLTR activity in response to LTB₄. Any of the assays described herein as suitable for identifying a modulator of BLTR receptor activity may be performed in the absence of a test compound to determine whether a polypeptide has the same essential character as LTRGW1.

Preferably a polypeptide with the same essential character as LTRGW1 enhances LTB₄ mediated activity of BLTR in a dose dependent manner. Typically, when cells are transfected with equal amounts by weight of BLTR and a polypeptide with the same essential character as LTRGW1, the maximal response of BLTR to LTB₄ is enhanced from 30 to 70 fold, preferably from 40 to 60 fold, more preferably from 45 to 55 fold by the LTRGW1 polypeptide.

Typically a polypeptide with the same essential character as LTRGW1 is capable of binding BLTR. Preferably binding of LTRGW1 and BLTR occurs when LTRGW1 is recruited to a BLTR-LTB₄ receptor complex. In this aspect, the LTRGW1 polypeptide may enhance BLTR activity, or indeed may not be active in this respect, and may be used to bind to BLTR to prevent BLTR from binding to active LTRGW1. An LTRGW1 polypeptide may form heteromultimers with a BLTR polypeptide, such that one or more LTRGW1 polypeptides complex with one or more BLTR polypeptides. Such multimers are termed LTRGW1/BLTR throughout the specification. Preferably, the heteromultimer is a heterodimer, comprising one LTRGW1 polypeptide and one BLTR polypeptide.

Binding of the LTRGW1 polypeptide to BLTR may be measured by any suitable means. For example, binding of an LTRGW1 polypeptide to BLTR may be measured by immunoprecipitating said LTRGW1 polypeptide and detecting co-immunoprecipitation of BLTR or by immunoprecipitating BLTR and detecting co-immunoprecipitation of said LTRGW1 polypeptide. Immunoprecipitation techniques are well known in the art. The binding assay may be carried out in the presence of a BLTR ligand, such as LTB₄ or leukotriene-B₄-3-aminopropylamide (LTB₄-APA). LTB4-APA may be crosslinked to BLTR (Goldman (1991) J. Immunol. 146, 2671-2677.

Alternatively, the interaction between LTRGW1 and BLTR may be monitored using fluorescence resonance energy transfer (FRET) (Guo et al., (1995) J. Biol. Chem. 270, 27562-27568) or bioluminescence resonance energy transfer (BRET) (Angers,S et al 2000 Proceedings of the National Academy of Sciences of the United States of America, 97, 3684-3689

In another aspect, a polypeptide with the same essential character as LTRGW1 may also be defined as one which binds to the same ligand as LTRGW1. This may be, for example, LTB₄, 12-epi-LTB₄, LTB₃, LTB5 LTD₄, LTE₄, LTC₄, or LTF₄. Preferably an LTRGW1 polypeptide will bind LTB₄. In this aspect, a polypeptide having the same essential character as LTRGW1 may be identified by monitoring for binding of leukotrienes, for example, using radiolabelled LTB₄ or other leukotrienes. Any of the assays described herein as suitable for identifying a modulator of LTRGW1 activity may be performed in the absence of a test compound to determine whether a polypeptide has the same essential character as LTRGW1.

Preferably a polypeptide with the same essential character as LTRGW1 enhances binding of LTB₄ to the membranes of cells co-expressing BLTR and the LTRGW1 polypeptide compared to cells expressing only BLTR or the LTRGW1 polypeptide. A typical assay for determining whether an LTRGW1 polypeptide enhances the binding of LTB₄ in this manner comprises preparing membranes from mammalian cells or Xenopus oocytes co-expressing the LTRGW1 polypeptide and BLTR, performing a scintillation proximity assay using wheat germ agglutinin beads and [³H]LTB₄ and comparing the binding data obtained to that obtained with membranes from cells expressing only BLTR or only LTRGW1 (Yokomizo et al. (1997) Nature 387, 620-624).

Preferably a polypeptide with the same essential character as LTRGW1 enhances LTB₄ binding from two to six fold, more preferably from four to five fold, or most preferably three fold in cells co-expressing the said polypeptide and BLTR compared to cells expressing BLTR alone. LTRGW1 may be used to discover modulators of the interaction between BLTR and LBT4

A full length protein is preferably one which includes a seven transmembrane region. Preferably, the full length receptor may couple to a G-protein to mediate intracellular responses.

Throughout the present specification the terms “BLTR polypeptide”, “BLTR receptor” and “BLTR” refer to the leukotriene-B₄ receptor polypeptide, comprising

(i) the amino acid sequence of SEQ ID NO: 8, or

(ii) A variant of (i) which is capable of binding LTB4; or

(iii) A fragment of (i) or (ii), which is capable of binding LTB4

A preferred fragment of BLTR has the amino acid sequence shown in SEQ ID NO: 6. The term “variant” and “fragment” in relation to BLTR refer to a polypeptide which has the same essential character or basic biological functionality as BLTR. The essential character of BLTR can be defined as follows: BLTR is a G-protein coupled leukotriene-B₄ receptor which is activated by LTB₄. LTB₄ activation of a BLTR polypeptide can be enhanced by LTRGW_{1.} Any of the assays described herein as suitable for identifying a modulator of BLTR receptor activity may be performed in the absence of a test compound to determine whether a polypeptide has the same essential character as BLTR.

A typical assay for determining whether the response of a BLTR polypeptide to LTB₄ is potentiated by LTRGW1 comprises co-expressing the BLTR polypeptide with LTRGW1 in mammalian cells, incubating cells with a calcium indicator dye, measuring the activity of the BLTR polypeptide in response to LTB₄ using a Fluorescence Imaging Plate Reader (FLIPR) and comparing the response to that obtained in cells expressing the BLTR polypeptide alone or the BLTR polypeptide and a different level of LTRGW1.

Preferably LTRGW1 enhances LTB₄ mediated activity of a polypeptide with the same essential character as BLTR in a dose dependent manner. Typically, when cells are transfected with equal amounts by weight of LTRGW1 and a polypeptide with the same essential character as BLTR, the maximal response of BLTR to LTB₄ is enhanced from 30 to 70 fold, preferably from 40 to 60 fold, more preferably from 45 to 55 fold by LTRGW1.

Typically a polypeptide with the same essential character as BLTR is capable of binding LTRGW1. Binding of the BLTR polypeptide to LTRGW1 may be measured by any suitable means. For example, binding of the BLTR polypeptide to LTRGW1 may be measured by immunoprecipitating said BLTR polypeptide and detecting co-immunoprecipitation of LTRGW1 or by immunoprecipitating LTRGW1 and detecting co-immunoprecipitation of said BLTR polypeptide. Immunoprecipitation techniques are well known in the art. The binding assay may be carried out in the presence of a BLTR ligand, such as LTB₄ or leukotriene-B₄-3-aminopropylamide (LTB₄-APA).

Alternatively, the interaction between LTRGW1 and BLTR may be monitored using fluorescence resonance energy transfer (FRET) (Guo et al., (1995) J. Biol. Chem. 270, 27562-27568) or bioluminescence resonance energy transfer (BRET) (Angers,S et al 2000 Proceedings of the National Academy of Sciences of the United States of America, 97, 3684-3689

In another aspect, a polypeptide with the same essential character as BLTR is one which binds to the same ligand as BLTR. Preferably a polypeptide with the same essential character as BLTR will bind LTB₄. In this aspect, a polypeptide having the same essential character as BLTR may be identified by monitoring for binding of a BLTR ligand, for example, using radiolabelled LTB₄. Typically binding of LTB₄ to BLTR is enhanced in the presence of LTRGW1. Preferably the affinity of BLTR for LTB₄ is enhanced by the recruitment of LTRGW1 to the BLTR-LTB₄ receptor complex.

Preferably a polypeptide with the same essential character as BLTR is capable of coupling to a G-protein.

The following description of variants and fragments of the LTRGW1 polypeptide applies also to variants and fragments of BLTR except that rather than being in relation to the amino acid sequences shown in SEQ ID NO: 2 and SEQ ID NO: 4, the sequence identities are in relation to the amino acid sequences shown in SEQ ID NO: 8 and SEQ ID NO: 6 and the basic biological functionality is that of the BLTR receptor.

Typically, polypeptides with more than about 65% identity preferably at least 80% or at least 90% and particularly preferably at least 95% at least 96% at least 97% at least 98% or at least 99% identity, with the amino acid sequences of SEQ ID NO: 2 or SEQ ID NO: 4 are considered as variants of LTRGW1, provided that they retain the basic biological functionality or essential charactre of the LTRGW1 polypeptide as herein defmed. Such variants may include allelic variants and the deletion, modification or addition of single amino acids or groups of amino acids within the protein sequence, as long as the peptide maintains the basic biological functionality of the LTRGW1 receptor.

Amino acid substitutions may be made, for example from 1, 2 or 3 to 10, 20 or 30 substitutions. The modified polypeptide generally retains activity as an LTRGW1 receptor. Conservative substitutions may be made, for example according to the following Table. Amino acids in the same block in the second column and preferably in the same line in the third column may be substituted for each other.

| | | |
|---|---|---|
| ALIPHATIC | Non-polar | G A P |
| | | I L V |
| | Polar-uncharged | C S T M |
| | | N Q |
| | Polar-charged | D E |
| | | K R |
| AROMATIC | | H F W Y |

Shorter polypeptide sequences are within the scope of the invention. For example, a peptide of at least 20 amino acids or up to 50, 60, 70, 80, 100, 150 or 200 amino acids in length is considered to fall within the scope of the invention as long as it demonstrates the basic biological functionality of LTRGW1. In particular, but not exclusively, this aspect of the invention encompasses the situation when the protein is a fragment of the complete protein sequence and may represent a ligand-binding region (N-terminal extracellular domain) or an effector binding region (C-terminal intracellular domain). Such fragments can be used to construct chimeric receptors preferably with another 7-transmembrane receptor, more preferably with another leukotriene-B₄ receptor. Such fragments can also be used to raise anti-LTRGW1 antibodies. In this embodiment the fragment may comprise an epitope of the LTRGW1 polypeptide and may otherwise not demonstrate the ligand binding or other properties of LTRGW1.

Polypeptides of the invention may be chemically modified, e.g. post-translationally modified. For example, they may be glycosylated or comprise modified amino acid residues. They may also be modified by the addition of histidine residues to assist their purification or by the addition of a signal sequence to promote insertion into the cell membrane. Polypeptides of the invention may be tagged to aid detection, for example using a VSV, HA, T7, myc or flag tag. Such modified polypeptides fall within the scope of the term “polypeptide” of the invention.

The invention also includes cells that have been modified to express an LTRGW1 polypeptide and which are also modified to express a BLTR polypeptide. Such cells include transient, or preferably stable higher eukaryotic cell lines, such as mammalian cells or insect cells, lower eukaryotic cells, such as yeast or prokaryotic cells such as bacterial cells. Particular examples of cells which may be modified by insertion of vectors encoding for LTRGW1 and BLTR polypeptides include mammalian HEK293T, CHO, HeLa and COS cells. Stable cell lines expressing LTRGW1 and BLTR may be isolated using a chemotaxis assay. Preferably the cell line selected will be one which is not only stable, but also allows for mature glycosylation and cell surface expression of a polypeptide. Expression may be achieved in transformed oocytes. A polypeptide of the invention may be expressed in cells of a transgenic non-human animal, preferably a mouse. A transgenic non-human animal expressing a polypeptide of the invention is included within the scope of the invention. A polypeptide of the invention may also be expressed in Xenopus laevis oocytes or melanophores, in particular for use in an assay of the invention.

It is also possible for the polypeptides of the invention to be transiently expressed in a host cell or on a membrane, such as for example in a baculovirus expression system. Such systems, which are adapted to express the polypeptides according to the invention, are also included within the scope of the present invention. Preferably such systems are adapted to co-express an LTRGW1 polypeptide and BLTR.

An important aspect of the present invention is the use of polypeptides according to the invention in screening methods to identify substances that may act as agonists or antagonists which may modulate leukotriene-B₄ receptor activity. This may take three forms

i) screening for substances that modulate LTRGW1's leukotriene activity,

ii) using LTRGW1 polypeptides to enhance the responsiveness of BLTR to LTB4 and hence screening for substances that modulate BLTR's leukotriene activity, or modulate the ability of LTRGW1 to enhance the activity of BLTR,

iii) screening for substances that modulate the interaction between LTRGW1 and BLTR.

The term modulators as used herein should be interpreted to mean substances that are agonists or antagonists of the interaction of either receptor and its respective ligands, or that upregulate or downregulate the interaction between LTRGW1 and BLTR. Preferably, modulators are antagonists of the LTRGW1 receptor, or are able to downregulate the interaction between LTRGW1 and BLTR.

To identify modulators of LTRGW1's leukotriene binding ability, any suitable form may be used for the assay. In general terms, such screening methods may involve contacting an LTRGW1 polypeptide with a test compound and then measuring receptor activity or may involve incubating an LTRGW1 polypeptide with a test substance and then detecting modulation of leukotriene activity at the LTRGW1 receptor. Agents which bind to the LTRGW1 polypeptides can also be identified by binding assays.

Modulator activity can be determined by contacting cells expressing an LTRGW1 polypeptide with a substance under investigation and by monitoring the effect mediated by the LTRGW1 polypeptides. The cells expressing the LTRGW1 polypeptide may be in vitro or in vivo. The LTRGW1 polypeptide may be naturally or recombinantly expressed. Preferably, the assay is carried out in vitro using cells expressing recombinant LTRGW1 polypeptide. Typically, LTRGW1 receptor activity can be monitored indirectly by measuring a Gi-coupled readout. Gᵢ coupled readout can typically be monitored using an electrophysiological method to determine the activity of G-protein regulated Ca²⁺ or K⁺ channels or by using a fluorescent dye to measure changed in intracellular Ca²⁺ levels. Other methods that can typically be used to monitor LTRGW1 receptor activity involved measuring levels of or activity of GTPγS, cAMP or chemotaxis. An assay of the invention may be carried out using a known leukotriene agonist or leukotriene antagonist to provide a comparison with a modulator under test.

For example, Kamohara et al (Journal of Biological Chemistry Vol. 275 Issue 35 pp 27000-27004, Oct. 7, 2000) have shown that leukotrienes LTB4, LTB3, LTB5 and 12-epi-LTB4 when binding to LTRGW1 cause inhibition of forskolin-stimulated intracellular cAMP accumulation, and further that LTB4 induces chemotaxis. This paper both demonstrates the functionality of LTRGW1 and indicates that such functional assays may be used to measure modulation of the leukotriene mediated activity of LTRGW1. Addition of a suspected modulator to such an assay allows determination of whether the functional response is increased or decreased, or whether there is no change. Yokomizo et al (Journal of Experimental Medicine Vol. 192, number 3 pp 421-432 Jul. 8, 2000)have also shown forskolin stimulated intracellular cAMP accumulation and chemotaxis when LTRGW1 is exposed to leukotrienes, and further demonstrate that LTB₄ increases cellular calcium, demonstrating that this is another suitable assay to measure modulator activity at the LTRGW1 receptor.

To identify a modulator of BLTR's leukotriene activity by using an LTRGW1 polypeptide, or to identify a modulator of LTRGW1's ability to bind to, and enhance the activity of, BLTR any suitable form may be used for the assay. In general terms, such screening methods may involve contacting an LTRGW1 polypeptide and a BLTR polypeptide, with a test substance and then measuring BLTR receptor activity or may involve incubating an LTRGW1 polypeptide and a BLTR polypeptide with a test substance and then detecting modulation of leukotriene activity at the BLTR receptor. Substances which bind to LTRGW1 or BLTR polypeptides can also be identified by binding assays. Preferably the assay may be carried out in a single well of a microtitre plate. Assay formats which allow high throughput screening are preferred.

Modulator activity can be determined by contacting cells co-expressing an LTRGW1 polypeptide and a BLTR polypeptide, with a substance under investigation and by monitoring the effect mediated by the BLTR receptor. To determine whether a test substance acts as an antagonist of LTB₄ at the BLTR/LTRGW1 receptor the effect of a test substance on the activation of BLTR by LTB₄ or another BLTR agonist may be monitored. A typical method for determining whether a test substrate acts as a BLTR agonist comprises monitoring stimulation of BLTR activity by contacting an LTRGW1 polypeptide and a BLTR polypeptide with a test substance and monitoring for BLTR activity. The cells expressing the polypeptide may be in vitro or in vivo. The LTRGW1 polypeptide and/or the BLTR polypeptide may be naturally or recombinantly expressed. Preferably, the assay is carried out in vitro using cells expressing recombinant BLTR polypeptide. More preferably, the cells express both recombinant LTRGW1 polypeptide and recombinant BLTR polypeptide.

Typically, receptor activity can be monitored indirectly by measuring a G_{q}/Gᵢ-coupled readout. G_{q}/Gᵢ coupled readout can typically be monitored using an electrophysiological method to determine the activity of G-protein regulated Ca²⁺ or K⁺ channels or by using a fluorescent dye to measure changed in intracellular Ca²⁺ levels. Other methods that can typically be used to monitor receptor activity involve measuring levels of or activity of GTPγS, cAMP or chemotaxis. An assay of the invention may be carried out using a known leukotriene-B₄ agonist or leukotriene-B₄ antagonist to provide a comparison with a modulator under test.

A standard assay for measuring activation of the Gᵢ family of G proteins is the GTP_{γ}S binding assay. Agonist binding to G protein-coupled receptors promotes the exchange of GTP for GDP bound to the α subunit of coupled heterotrimeric G proteins. Binding of the poorly hydrolysable GTP analogue, [³⁵S]GTP_{γ}S, to membranes has been used extensively as a functional assay to measure agonism at a wide variety of receptors. Furthermore, the assay is largely restricted to measuring function of receptors coupled to the Gᵢ family of G proteins due to their ability to bind and hydrolyse guanine nucleotide at significantly higher rates than members of the G_{q}, Gₛ, and G₁₂ families. See Wieland and Jakobs, Methods Enzymol. 237, 3-13, 1994.

G protein coupled receptors (GPCRs) have been shown to activate MAPK signalling pathways. Host cells overexpressing the LTRGW1 and BLTR polypeptides with MAPK reporter genes may be utilised as assays for receptor activation or inhibition. For example, yeast assays may be used to screen for agents that modulate the activity of an LTRGW1/BLTR receptor. A typical yeast assay involves heterologously expressing an LTRGW1/BLTR receptor in a modified yeast strain containing multiple reporter genes, typically FUS1-HIS3 and FUS1-lacZ, each linked to an endogenous MAPK cascade-based signal transduction pathway. This pathway is normally linked to pheromone receptors, but can be coupled to foreign receptors by replacement of the yeast G protein with yeast/mammalian G protein chimeras. Strains may also contain further gene deletions, such as deletions of SST2 and FAR1, to potentiate the assay. Ligand activation of the heterologous receptor can be monitored for example either as cell growth in the absence of histidine or with a suitable substrate such as beta-galactosidase (lacZ).

Alternatively melanophore assays may be used to screen for activators of an LTRGW1/BLTR receptor. An LTRGW1/BLTR receptor can be heterologously expressed in Xenopus laevis melanophores and their activation can be measured by either melanosome dispersion or aggregation. Basically, melanosome dispersion is promoted by activation of adenylate cyclase or phospholipase C, i.e. Gₛ, and G_{q} mediated signalling respectively, whereas aggregation results from activation of Gᵢ-protein resulting in inhibition of adenylate cyclase. Hence, ligand activation of the heterologous receptor can be measured simply by measuring the change in light transmittance through the cells or by imaging the cell response.

Assays may also be carried out by incubating a cell expressing a BLTR polypeptide and an LTRGW₁ polypeptide with a test substance in the presence of neutrophils or other cells of the immune system. Chemotaxis of the neutrophils associated with stimulation of the receptor of the invention can be monitored. Similarly, neutrophil degranulation and release of mediators, enzymes and superoxides from neutrophils can be measured to monitor or assess activation of the BLTR/LTRGW1 receptor in the presence of a test substance.

Preferably, control experiments are carried out on cells which do not express LTRGW1 to establish whether the observed responses are the result of activation or inhibition of the polypeptide. More preferably, control experiments are carried out on cells which express BLTR but which do not express LTRGW1.

All assays may be carried out utilising cells expressing only BLTR and/or only LTRGW1 and the results of those experiments may be compared to the results of parallel experiments on cells expressing both BLTR and LTRGW1 to ensure any effects of a test substance are dependent on the presence of LTRGW1 in the cells.

The binding of a modulator to an LTRGW1 polypeptide or BLTR polypeptide can also be determined directly. For example, a radiolabelled test substance can be incubated with LTRGW1 polypeptide or BLTR polypeptide and binding of the test substance to the polypeptide can be monitored. Typically, the radiolabelled test substance can be incubated with cell membranes or cells containing the polypeptide until equilibrium is reached. The membranes can then be separated from a non-bound test substance and dissolved in scintillation fluid to allow the radioactive content to be determined by scintillation counting. Non-specific binding of the test substance may also be determined by repeating the experiment in the presence of a saturating concentration of a non-radioactive ligand. Preferably such binding assays are carried out on cells co-expressing an LTRGW1 polypeptide and a BLTR polypeptide. Preferably the binding of a test substance to cells co-expressing an LTRGW1 polypeptide and a BLTR polypeptide is compared to the binding of a test substance to cells expressing only BLTR and/or to cells expressing only an LTRGW1 polypeptide.

Alternatively, ligand binding may be monitored by binding a fluorescent ligand such as LTB₄-APA-fluoroscein to cells expressing the polypeptides of interest and detecting bound ligand by fluorescence activated cell sorting (FACS).

A test substance may modulate leukotriene mediated activity by disrupting the interaction between LTRGW1 and BLTR. An assay which monitors the interaction between LTRGW1 and BLTR may be used to screen for substances that modulate leukotriene activity. For example, an immunoprecipitation assay, a pull-down assay, an affinity-purification assay or a fluorescence resonance energy transfer (FRET) assay may be used to determine the effect of a test substance on the interaction between BLTR and LTRGW1. An LTRGW1 polypeptide for use in such an assay is capable of binding to BLTR but may, or may not, possess other essential characteristics of LTRGW1. A BLTR polypeptide for use in such an assay is capable of binding to LTRGW1 but may, or may not, possess other essential characteristics of BLTR.

Suitable test substances which can be tested in the above assays include combinatorial libraries, defined chemical entities, peptide and peptide mimetics, oligonucleotides and natural product libraries, such as display (e.g. phase display libraries) and antibody products.

Test substances may be used in an initial screen of, for example, 10 substances per reaction, and the substances of these batches which show inhibition or activation tested individually. Test substances may be used at a concentration of from 1 nM to 1000 μM, preferably from 1 μM to 100 μM, more preferably from 1 μM to 10 μM.

Another aspect of the present invention is the use of the substances that have been identified by screening techniques referred to above in the treatment or prophylaxis of disorders which are responsive to regulation of leukotriene-B₄ receptor activity. Typically modulators useful in the therapeutic or prophylactic treatment of such disorders are inhibitors of leukotriene-B₄ receptor activity. In particular, such substances may be used in the treatment of acute and chronic inflammatory diseases, such as asthma, chronic obstructive pulmonary disease (COPD), allergic rhinitis, hayfever, immune deficiency disorder, AIDS, rheumatoid arthritis, multiple sclerosis, leukaemia, myesthenia gravis, graves disease, systemic lupus erythematosus, inflammatory bowel disease, encephalomyelitis, psoriasis, atopic dermatitis, septic shock, stroke, ischaemia reperfusion injury and cardiovascular diseases. Preferably, such substances are used in the treatment of asthma, COPD, Rheumatoid arthritis and psoriasis. Particularly preferred is when such substances are used in the treatment of asthma. It is to be understood that mention of these specific disorders is by way of example only and is not intended to be limiting on the scope of the invention as described.

The substances identified according to the screening methods outlined above may be formulated with standard pharmaceutically acceptable carriers and/or excipients as is routine in the pharmaceutical art, and as fully described in Remington's Pharmaceutical Sciences, Mack Publishing Company, Eastern Pennsylvania 17^{th} Ed. 1985, the disclosure of which is included herein of its entirety by way of reference. The carrier or excipient may be an isotonic saline solution but will depend more generally upon the particular agent concerned and the route by which the agent is to be administered.

The substances may be administered by enteral or parenteral routes such as via oral, buccal, anal, pulmonary, intravenous, intra-arterial, intramuscular, intraperitoneal, topical or other appropriate administration routes. A therapeutically effective amount of a modulator is administered to a patient. The dose of a modulator may be determined according to various parameters and especially according to the substance used; the age, weight and condition of the patient to be treated; the route of administration; and the required regimen. A physician will be able to determine the required route of administration and dosage for any particular patient. A typical daily dose is from about 0.1 to 50 mg per kg of body weight, according to the activity of the specific modulator, the age, weight and conditions of the subject to be treated, the type and severity of the degeneration and the frequency and route of administration. Preferably, daily dosage levels are from 5 mg to 2 g.

Alternatively substances which down-regulate LTRGW1 expression or nucleic acid encoding a polypeptide, preferably an LTRGW1 variant polypeptide, which inhibits the function of LTRGW1 may be administered to the mammal. Nucleic acid, such as RNA or DNA, preferably DNA, is provided in the form of a vector, which may be expressed in the cells of a human or other mammal under treatment. Preferably such down-regulation or expression following nucleic acid administration will inhibit LTRGW1 mediated potentiation of BLTR activity.

Nucleic acid encoding the LTRGW1 or variant polypeptide may be administered to a human or other mammal by any available technique. For example, the nucleic acid may be introduced by injection, preferably intradermally, subcutaneously or intramuscularly. Alternatively, the nucleic acid may be delivered directly across the skin using a nucleic acid delivery device such as particle-mediated gene delivery. The nucleic acid may be administered topically to the skin, or to the mucosal surfaces for example by intranasal, oral, intravaginal, intrarectal administration.

Uptake of nucleic acid constructs may be enhanced by several known transfection techniques, for example those including the use of transfection agents. Examples of these agents includes cationic agents, for example, calcium phosphate and DEAE-Dextran and lipofectants, for example, lipofectam and transfectam. The dosage of the nucleic acid to be administered can be altered. Typically the nucleic acid is administered in the range of 1 pg to 1 mg, preferably to 1 pg to 10 μg nucleic acid for particle mediated gene delivery and 10 μg to 1 mg for other routes.

Polynucleotides encoding LTRGW1 or a variant polypeptide can also be used to identify mutation(s) in LTRGW1 genes which may be implicated in human disorders. Identification of such mutation(s) may be used to assist in diagnosis of acute and chronic inflammatory diseases, such as asthma, chronic obstructive pulmonary disease (COPD), allergic rhinitis, hayfever, immune deficiency disorder, AIDS, rheumatoid arthritis, multiple sclerosis, leukaemia, myesthenia gravis, graves disease, systemic lupus erythematosus, inflammatory bowel disease, encephalomyelitis, psoriasis, atopic dermatitis, septic shock, stroke, ischaemia reperfusion injury, cardiovascular diseases or susceptibility to such disorders and in assessing the physiology of such disorders. Preferably, the disorder is asthma, COPD, rheumatoid arthritis or psoriasis.

Antibodies (either polyclonal or preferably monoclonal antibodies, chimeric, single chain, Fab fragments) which are specific for the LTRGW1 polypeptide or a variant thereof can be generated. Such antibodies may for example be useful in purification, isolation or screening methods involving immunoprecipitation techniques and may be used as tools to elucidate further the function of LTRGW1 or a variant thereof, or indeed as therapeutic agents in their own right. Such antibodies may be used to block ligand binding to the receptor. A variety of protocols for competitive binding or immunoradiometric assays to determine the specific binding capability of an antibody are well known in the art (see for example Maddox et al, J. Exp. Med. 158, 1211 et seq, 1993).

The following Examples illustrate the invention.

### EXAMPLE 1

LTRGW1 Potentiation of BLTR Activity in Response to LTB₄

The 388 amino acids (aa) encoding for LTRGW1 peptide were aligned to other known seven transmembrane proteins and putative transmembrane domains were identified as follows: TM1 aa 55-77, TM2 aa 91-113, TM3 aa 124-148, TM4 aa 168-187, TM6 aa 256-277, TM7 aa 305-324. Hydrophobicity plot analysis confirmed these areas as putative transmembrane domains. LTRGW1 does not contain a signal peptide at its amino terminal end. The closest protein is the BLT receptor (BLTR) with 50% similarity and 45% identity throughout their length. LTRGW1 gene is localised in the near proximity of the already described BLTR suggesting that it could have been evolved as a result of gene duplication. Indeed they share a 61% identity at the DNA level over a stretch of 885 bases. Expression constructs were generated from both putative methionine (position 1 or 32 in SEQ ID NO: 1) using the following 5′ end primers respectively:

GGAATTCGCACCATGGCACCTTCTCATCGGGCATCACAG (LL1) and

GGAATTCGCACCATGTCGGTCTGCTACCGTCCCCCA (LL2).

Genomic DNA was used as template in PCR reaction using either LL1 or LL2 primer together with a 3′ end primer with the following sequence:

GCTCTAGATCAAAGGTCCCATTCCGGACCGTCCTTC (LL6).

PCR products were digested with EcoRI and XbaI and subcloned into pcDNA3 (pLTRGW1-1 and pLTRGW1-32 for corresponding methionines). pLTRGW1-1 and pLTRGW1-32 were fully sequenced and their pharmacological properties when co-expressed with BLTR were assessed in Xenopus laevis oocyte and CHO over-expression systems.

10 mg total DNA was transfected into CHO cells using the following transfection mix: 5 μg BLTR, pcDNA3 as needed to keep constant the amount of total DNA used, 1 μg pCMV-luciferase and BLTR-2 at various concentration. Responses to LTB₄ (between 10⁻¹⁰ and 10⁻⁷ M) were measured using a FLIPR assay (Fluorescence Imaging Plate Reader—Molecular Devices) and values were normalised relative to the luciferase reading. The results are shown in Table 1. Under these particular experimental conditions LTRGW1 expressed alone does not induce a detectable mobilisation of intracellular calcium in response to LTB₄. However, as shown by Kamohara et al (Journal of Biological Chemistry Vol. 275 Issue 35 pp 27000-27004, Jul. 10, 2000) and Yokomizo et al (Journal of Experimental Medicine Vol. 192, number 3 pp 421-432 Aug. 7, 2000), it is possible to detect responses such as inhibition of forskolin-stimulaated intracellular cAMP accumulation, and chemotaxis, indicating that LTRGW1 is responsive to leukotrienes. Yokomizo et al also manage to show that LTB₄ increases intracellular calcium.

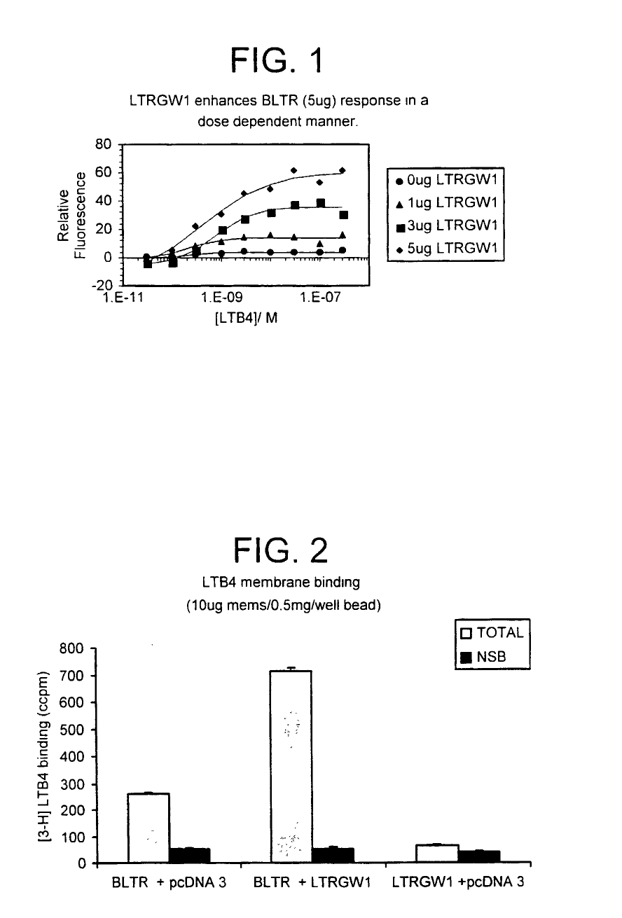

The response of BLTR to LTB₄ is increased when LTRGW1 is co-expressed with BLTR. The size of the increased response is proportional to the amount of LTRGW1 in the transfection mix. This enhancement of LTB₄ activation of BLTR in the presence of LTRGW1 is illustrated in FIG. 1.

TABLE 1

| CHO cells transfected with | Response to LTB4 |
|---|---|
| Mock transfected | Inactive |
| BLTR | Active |
| LTRGW1₍₂₇₅₎ | Inactive |
| LTRGW1₍₂₇₄₎ | Inactive |
| LTRGW1₍₂₇₆₎ | Inactive |
| [Signal Peptide-FLAG-LTRGW1₍₂₇₆₎] | [FACS showed surface |
| | localisation] |
| BLTR/LTRGW1₍₂₇₅₎ | Active |
| 1 μg | + |
| BLTR/LTRGW1₍₂₇₅₎ | Active |
| 3 μg | ++ |
| BLTR/LTRGW1₍₂₇₅₎ | Active |
| 5 μg | +++ |
| BLTR/LTRGW1₍₂₇₆₎ | Active |

### EXAMPLE 2

Enhanced Binding of LTB₄ to Membranes of Cells Co-Expressing LTRGW1 and BLTR

CHO cells were transfected with DNA as described in Example 1. [³H] LTB₄ binding to membrane preparations from transfected cells was measured using wheat germ agglutinin beads in a scintillation proximity assay. The assay was carried out in 50: g Hepes 20: g MgCl₂ in a total assay volume of 100: g. [³H] LTB₄ was used to measure total binding. [³H] LTB₄ was then displaced with unlabelled LTB₄ to measure non-specific binding (NSB). The binding of LTB₄ to cells expressing BLTR cells co-expressing BLTR and LTRGW1 and cells expressing LTRGW1 is illustrated in FIG. 2.

### EXAMPLE 3

Tissue Distribution of LTRGW1 and BLTR

LTRGW1 mRNA tissue distribution was studied using the following oligonucleotides as forward, reverse and probe primers respectively:

5′GCGCGAGCGGGAACTA-3′

5′AGCGGTGAAGACGTAGAGCAC-3′

5′CCTTGGCCTTCTTCAGTTCTAGCGTCAA-3′

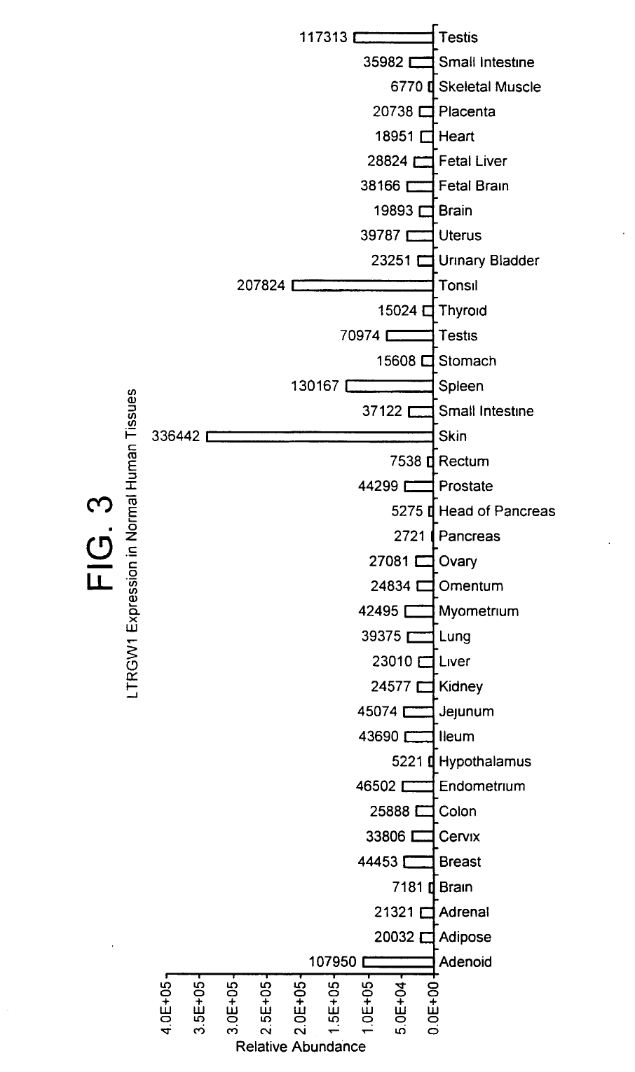

using Taqman™ PCR analysis (P. E. Biosystems). The results of this analysis on normal human tissues is shown in FIG. 3.

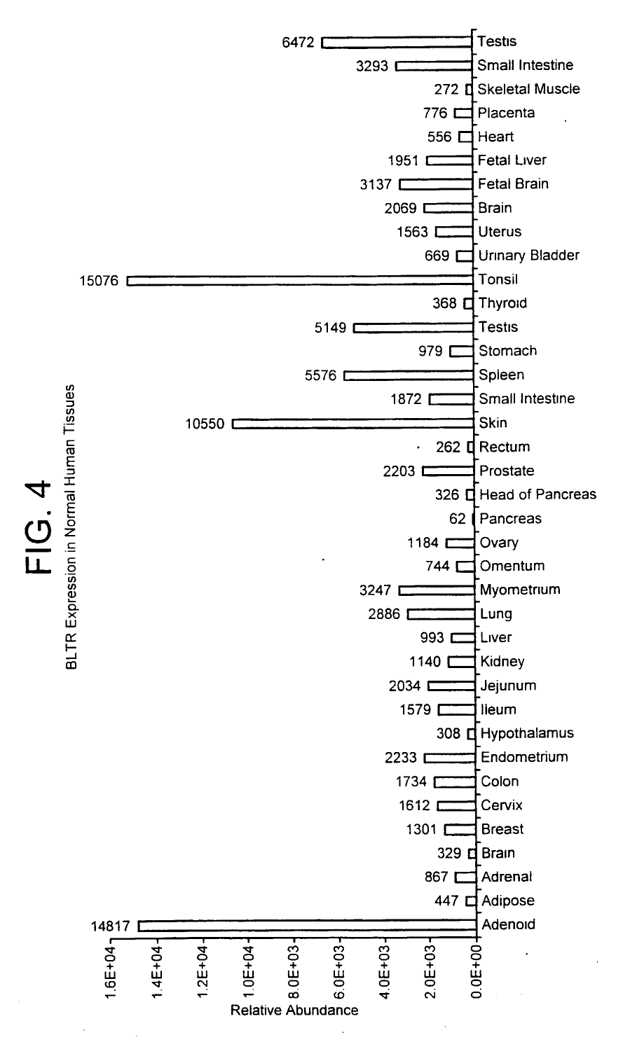

BLTR mRNA tissue distribution was also studied using Taqman™ PCR analysis. The tissue distribution of BLTR mRNA is shown in FIG. 4.

LTRGW1 and BLTR have an identical tissue distribution. Both appear to be ubiquitously expressed, with highest levels in skin, tonsil, spleen and adenoid.

### EXAMPLE 4

Comparison of Presence of Full Length Transcripts of BLTR and LTRGW1.

The presence of full length transcripts of the short and long forms of both BLTR and LTRGW1 was analysed in the spleen, testis and skin. The control was no DNA. The following primer sets were used:

| | | |
|---|---|---|
| LTRGW1 long: | LL1 | ATGGCACCTTCTCATCGGGCATCACAG |
| | LL6b | TCAAAGGTCCCATTCCGGACCGTCCTTC |
| LTRGW1 short: | LL2 | ATGTCGGTCTGCTACCGTGGGGGA |
| | LL6b | As above |
| BLTR long: | BLTR17 | ATGGCGTCAGGAAACCCTTGGTCCTC |
| | BLTR15 | CTAGTTCAGTTCGTTTAACTTGAGAGGGC |
| BLTR short: | BLTR4 | AACACTACATCTTCTGCAGCACCCCCCT |
| | BLTR15 | As above |

Spleen and Testis libraries were from Life Technologies (cat.No 10425-015 and 10426-103 respectively) Skin library was from Invitrogen (Cat. No. A900-14)

75 ng of DNA from each library was used in the PCR reaction, and 35 cycles of 94° C. for 60 seconds, 62° C. for 60 seconds, 70° C. for 120 seconds were carried out.

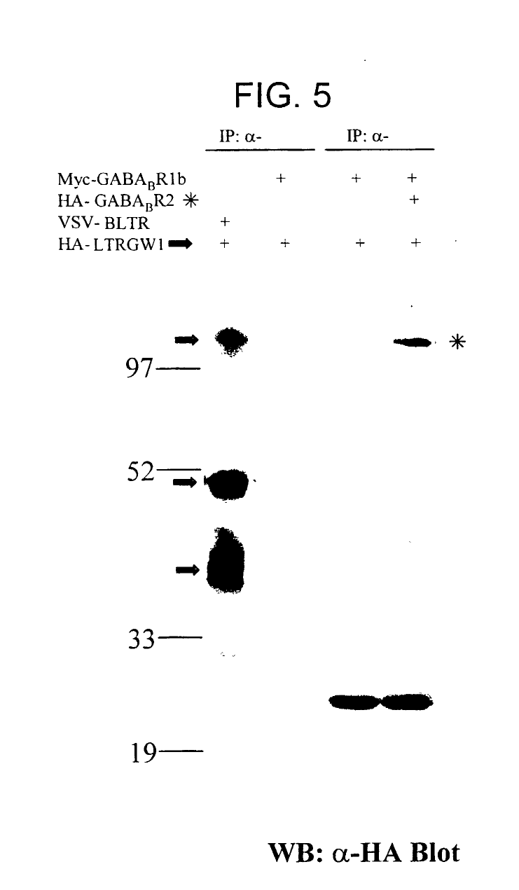

The results can be seen in FIG. 5. They show that cDNAs for both BLTR and LTRGW1 are present in all the tissues tested, further confirming the overlapping distribution suggested by the results of example 3. Interestingly, LTRGW1 is represented by the short form (Seq ID #4) only.

### EXAMPLE 5

Immunoprecipitation of BLTR and LTRGW1

To test whether BLTR and LTRGW1 form heterodimers, immunoprecipitation experiments were carried out. Transiently transfected CHO cells were harvested from 60 mm culture dishes. Cells from each dish were resuspended in 1 ml of 50 mM Tris-HCl, 150 mM NaCl, 1% (v/v) Nonidet® P40, 0.5% (w/v) sodium deoxycholate, pH 7.5 (lysis buffer) supplemented with Complete™ protease inhibitor cocktail tablets (1 tablet/25 ml) (Roche). Cell lysis and membrane protein solubilisation was achieved by passage through a 25-guage needle followed by gentle mixing for 30 min at 4° C. Insoluble debris was removed by microcentrifugation at 16,000 g for 15 min at 4° C. and the supernatant was precleared by incubating with 50 μl of Protein A-agarose (Roche) for 3 h at 4° C. on a helical wheel to reduce background caused by non-specific adsorption of cellular proteins. The solubilised supernatant was then divided into 2×500 ml aliquots and 20 ml of either BLTR (221), BLTR2 (287) or Myc antisera was added to each. Immunoprecipitation was allowed to proceed for 1 h at 4° C. on a helical wheel prior to the addition of 50 μl of Protein A-agarose suspension. Capture of immune complexes was progressed overnight at 4° C. on a helical wheel. Complexes were then collected by microcentrifugation 12,000 g for 1 min at 4° C. and supernatant was discarded. Beads were then washed by gentle resuspension and agitation sequentially in 1 ml of 50 mM Tris-HCl, pH 7.5, 500 mM NaCl, 0.1% (v/v) Nonidet® P40 and 0.05% (w/v) sodium deoxycholate followed by 1 ml of 50 mM Tris-HCl, pH 7.5, 0.1% (v/v) Nonidet^{\dot{O}} P40 and 0.05% (w/v) sodium deoxycholate. Immunoprecipitated proteins were released from Protein A-agarose by incubation in 30 μl of SDS-PAGE sample buffer at 70° C. for 10 min and analysed by SDS-PAGE followed by immunoblotting.

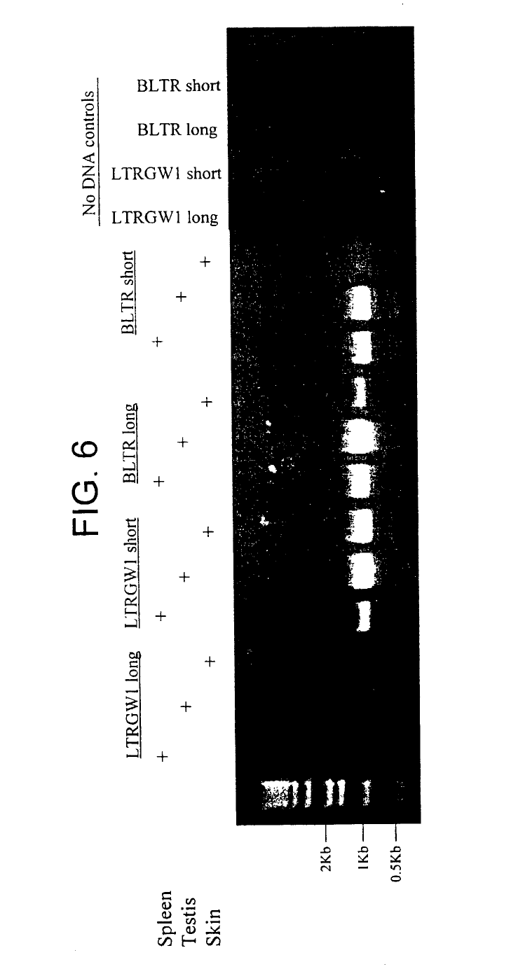

The results can be seen in FIG. 6. This experiment clearly demonstrates that BLTR and LTRGW1 co-precipitate, indicating that they have formed a dimer. This heterodimerisation was specific for the two receptors, since immunoprecipitation of GABAb, an unrelated G-protein coupled receptor did not pull down either leukotriene receptor when co-expressed. Furthermore, this experiment also demonstrates that BLTR and LTRGW1 receptors heterodimerise following overexpression even in the absence of ligand stimulation.

### EXAMPLE 6

Screening for Substances which Exhibit Protein Modulating Activity

(i) Transfection of Cells

Mammalian cells, such as HEK293, CHO or COS7 cells or Xenopus laevis oocytes over-expressing either a BLTR polypeptide, an LTRGW1 polypeptide, or both a BLTR polypeptide and an LTRGW1 polypeptide, are generated for use in the assays.

For example, Xenopus oocyte expression may be determined as follows. Adult female Xenopus laevis (Blades Biologicals) are anaesthetised using 0.2% tricaine (3-aminobenzoic acid ethyl ester), killed and the ovaries rapidly removed. Oocytes are then de-folliculated by collagenase digestion (Sigma type I, 1.5 mg ml⁻¹) in divalent cation-free OR2 solution (82.5 mM NaCl, 2.5 mM KCl, 1.2 mM NaH₂PO₄, 5 mM HEPES; pH 7.5 at 25° C.). Single stage V and VI oocytes are transferred to ND96 solution (96 mM NaCl, 2 mM KCl, 1 mM MgCl₂, 5 mM HEPES, 2.5 mM sodium pyruvate; pH 7.5 at 25° C.) which contains ⁵⁰ μg ml⁻¹ gentamycin and stored at 18° C.

LTRGW1 DNA and/or BLRT DNA (in pcDNA₃, Invitrogen) is linearised and transcribed to RNA using T7 (Promega Wizard kit). m′G(5′)pp(5′)GTP capped cRNA is injected into oocytes (20-50 ng per oocyte) and whole-cell currents are recorded using two-microelectrode voltage-clamp (Geneclamp amplifier, Axon instruments Inc.) 3 to 7 days post-RNA injection. Microelectrodes have a resistance of 0.5 to 2 MΩ when filled with 3M KCl.

(ii) Ligand Binding

Cells are transiently transfected with both tagged and untagged polypeptides. The binding of various concentrations of [³H] LTB₄ to membranes derived from cells overexpressing the relevant polypeptides is measured and normalised to the level of expression of tagged receptors. Non-specific binding of [³H]LTB₄ is determined by monitoring [³H]LTB₄ binding in the presence of non-radtioactive LTB₄.

Test substances are screened for their ability to displace [³H]LTB₄ from BLTR, LTRGW1 and/or BLTR/LTRGW1 receptors by repeating the experiment in the presence of non-radioactive test substance. (See Yokomizo T et al. 1997 Nature 387, 620-624).

Alternatively, ligand binding may be monitored by binding a fluorescent ligand such as LTB₄-APA-fluoroscein to cells expressing the polypeptides of interest and detecting bound ligand by fluorescence activated cell sorting (FACS).

(iii) Ca²⁺ Immobilisation (FLIPR)

96 and 384 well plate, high throughput screens (HTS) are employed using fluorescence based calcium indicator molecules, including but not limited to dyes such as Fura-2, Fura-Red, Fluo 3 and Fluo 4 (Molecular Probes). Secondary screening involves the same technology.

A screening assay may be conducted as follows. Mammalian cells stably over-expressing the protein(s) are cultured in black wall, clear bottom, tissue culture coated 96 or 384 well plates with a volume of 100 μl cell culture medium in each well 3 days before use in a FLIPR assay. Cells are incubated with 4 μM FLUO-3AM at 30° C. in 5%CO₂ for 90 mins and then washed once in Tyrodes buffer containing 3 mM probenecid. Basal fluorescence is determined prior to substance additions. The protein is activated upon the addition of a known agonist such as LTB₄. Activation results in an increase in intracellular calcium which can be measured directly in the FLIPR. For antagonist studies, substances are preincubated with the cells for 4 minutes following dye loading and washing and fluorescence is measured for 4 minutes. Agonists are then added and cell fluorescence is measured for a further 1 minute.

Transiently transfected CHO-Gα and wild type CHO cells are ideal for such experiments. Results can be compared to results of parallel experiments run in the presence of petussis toxin (PTX).

(iv) Accumulation of cAMP

Following leukotriene stimulation, cyclic AMP accumulation can be measured in forskolin stimulated cells such as CHO-Gα and wild-type CHO cells transfected with the LTRGW1 receptor either directly, by SPA assay, or indirectly by monitoring the expression of co-transfected reporter gene, the expression of which will be controlled by cyclic AMP response elements. Results are compared to parallel experiments run in the presence of PTX.

(v) Chemotaxis

A typical chemotaxis assay will measure the movement of LTRGW1 and BLTR transfected cells such as CHO cells through a polycarbonate filter with 8-μm pores towards the side in contact with the leukotriene ligand (Yokomizo T, et al 1997 Nature, 387, 620-624).

(vi) Fluorescence Resonance Energy Transfer (FRET)

The association of BLTR polypeptides and LTRGW1 polypeptides may be monitored using FRET. The polypeptides are co-expressed in cells and may be labelled with a donor probe, fluorescein or with an acceptor carbocyanine probe (Cy3). A typical FRET assay utilises cells co-expressing VSV-tagged BLTR and FLAG-tagged LTRGW1. The cells are stained with Cy3-labelled anti-VSV antibody and biotin-labelled anti-FLAG antibody and then with fluoroscein-streptavidin. FACS analysis of FRET between BLTR and LTRGW1 is then carried out in the absence of stimulation and/or following stimulation with LTB₄ or LTB₄-APA.

Alternatively, a typical FRET assay may measure FRET between LTB₄-APA-fluorescein and flag-tagged LTRGW1 stained with cy3 conjugated anti-FLAG antibody in cells expressing BLTR and flag-tagged LTRGW1.

(vii) Tertiary Screening

Tertiary screens involve the study of modulators in rat, mouse and guinea-pig models of disease relevant to the target.

1. A method for the identification of a compound which modulates leukotriene B₄ like (LTRGW1) receptor activity, which method comprises contacting an LTRGW1 polypeptide comprising:
i) The amino acid sequence of SEQ ID #2, or
ii) A variant of (i) which is capable of binding leukotrienes; or
iii) A fragment of (i) or (ii) which is capable of binding leukotrienes.
with a test compound in the presence of a leukotriene such as LTB₄, LTD₄, LTE₄, LTC₄, and LTT₄.

2. A method according to claim 1 wherein the variant (ii) has at least 80% identity to the seq of SEQ ID#2.

3. A method according to claim 1 or 2 which comprises monitoring the interaction between the LTRGW1 polypeptide and the leukotriene.

4. A method according to any preceeding claim wherein the LTRGW1 polypeptide is expressed in a cell.

5. Use of an LTRGW1 polypeptide as defined in claim 1 or 2 to enhance the leukotriene B₄ Receptor (BLTR) response to leukotrienes.

6. An isolated heterodimer comprising an LTRGW1 polypeptide as defined in claim 1 or 2 and a BLTR polypeptide comprising:
i) The amino acid sequence of SEQ ID #8, or
ii) A variant of (i) which is capable of binding leukotrienes or
iii) A fragment of (i) or (ii) which is capable of binding leukotrienes.

7. A method for increasing the responsiveness of a screen for identification of a a substance that modulates the activity of BLTR, comprising the addition to said screen of an LTRGW1 polypeptide as defined in claim 1 or 2.

8. A method for identification of a substance that modulates BLTR activity, which method comprises contacting an LTRGW1 polypeptide as defined in claim 1 or 2, and a BLTR polypeptide as defined in claim 6, with a test substance and monitoring for LTB₄ binding to the said polypeptides.

9. A method for identification of a substance that modulates BLTR receptor activity, which method comprises contacting an LTRGW1 polypeptide as defined in claim 1 or 2, and a BLTR polypeptide as defined in claim 6, with LTB₄ in the presence of a test substance and monitoring for BLTR activity.

10. A method according to claim 8 or 9 which comprises monitoring the activation of a G-protein.

11. A method for identification of a substance that modulates BLTR activity, which method comprises:
(i) providing
(a) an LTRGW1 polypeptide as defined in claim 1 or 2; and
(b) a BLTR polypeptide as defined in claim 6; and
(c) a test substance
under conditions that would permit the interaction of (a) and (b) in the absence of (c);
(ii) monitoring the interaction between (a) and (b); and
(iii) determining whether (c) modulates the interaction between (a) and (b) and thereby determining whether the test substance is a modulator of BLTR receptor activity.

12. A substance identified by a method according to any one of claims 1 to 4, 8, 9, 10 or 11.

13. A method of treating a subject having a disorder that is responsive to modulation of LTRGW1 or BLTR activity, which method comprises administering to said subject an effective amount of a substance according to claim 12.

14. A method according to claim 13 wherein said substance is an antagonist of LTRGW1 or BLTR.

15. A method of treating a subject having a disorder that is responsive to modulation of the interaction between LTRGW1 and BLTR, which method comprises administering to said subject an effective amount of a substance according to claim 14.

16. A method according to claim 15 wherein said substance downregulates the interaction between LTRGW1 and BLTR.

17. Use of a substance as defined in claim 12 in the manufacture of a medicament for treatment or prophylaxis of a disorder that is responsive to stimulation or modulation of LTRGW1 or BLTR activity.

18. Use according to claim 17 wherein said substance is an antagonist of LTRGW1 or BLTR

19. Use of a substance as defined in claim 12 in the manufacture of a medicament for treatment or prophylaxis of a disorder that is responsive to stimulation or modulation of the interaction between BLTR and LTRGW1

20. Use according to claim 19 wherein said substance downregulates the interaction between BLTR and LTRGW1.

21. A method according to any of claims 13 to 16, or a use according to any of claims 17 to 20 wherein the disorder is an acute or chronic inflammatory disease, asthma, chronic obstructive pulmonary disease or psoriasis.

22. A method of treating a patient with a respiratory disorder, said method comprising the administration of a therapeutically effective amount of an antagonist of LTRGW1

23. A method of treating a respiratory disorder, said method comprising the administration to a patient of a therapeutically effective amount of a substance that downregulates the interaction between LTRGW1 and BLTR, hence decreasing the response of BLTR to its ligand.

24. Use of a therapeutically effective amount of an antagonist of LTRGW1 in the manufacture of a medicament for the treatment or prophylaxis of respiratory diseases.

25. Use of an effective amount of a substance that downregulates the interaction between LTRGW1 and BLTR in the manufacture of a medicament for the treatment or prophylaxis of respiratory disorders.

26. A method according to claim 22 or 23, or a use according to claim 24 or 25 wherein said respiratory disorder is asthma or chronic obstructive pulmonary disorder.


SEQUENCE LISTING (8 sequences)

SEQ ID NO: 1 - DNA, 1170 bp, Homo sapiens
  CDS (1)..(1170)
atggcaccttctcatcgggcatcacaggtggggttttgccccacccctgaacgccctctgtggcgccttccacccacctgtaggcccagaaggatgtcggtctgctaccgtcccccagggaacgagacactgctgagctggaagacttcgcgggccacaggcacagccttcctgctgctggcggcgctgctggggctgcctggcaacggcttcgtggtgtggagcttggcgggctggcggcctgcacgggggcgaccgctggcggccacgcttgtgctgcacctggcgctggccgacggcgcggtgctgctgctcacgccgctctttgtggccttcctgacccggcaggcctggccgctgggccaggcgggctgcaaggcggtgtactacgtgtgcgcgctcagcatgtacgccagcgtgctgctcaccggcctgctcagcctgcagcgctgcctcgcagtcacccgccccttcctggcgcctcggctgcgcagcccggccctggcccgccgcctgctgctggcggtctggctggccgccctgttgctcgccgtcccggccgccgtctaccgccacctgtggagggaccgcgtatgccagctgtgccacccgtcgccggtccacgccgccgcccacctgagcctggagactctgaccgctttcgtgcttcctttcgggctgatgctcggctgctacagcgtgacgctggcacggctgcggggcgcccgctggggctccgggcggcacggggcgcgggtgggccggctggtgagcgccatcgtgcttgccttcggcttgctctgggccccctaccacgcagtcaaccttctgcaggcggtcgcagcgctggctccaccggaaggggccttggcgaagctgggcggagccggccaggcggcgcgagcgggaactacggccttggccttcttcagttctagcgtcaacccggtgctctacgtcttcaccgctggagatctgctgccccgggcaggtccccgtttcctcacgcggctcttcgaaggctctggggaggcccgagggggcggccgctctagggaagggaccatggagctccgaactacccctcagctgaaagtggtggggcagggccgcggcaatggagacccggggggtgggatggagaaggacggtccggaatgggacctttga

SEQ ID NO: 2 - protein, 389 aa, Homo sapiens
MAPSHRASQVGFCPTPERPLWRLPPTCRPRRMSVCYRPPGNETLLSWKTSRATGTAFLLLAALLGLPGNGFVVWSLAGWRPARGRPLAATLVLHLALADGAVLLLTPLFVAFLTRQAWPLGQAGCKAVYYVCALSMYASVLLTGLLSLQRCLAVTRPFLAPRLRSPALARRLLLAVWLAALLLAVPAAVYRHLWRDRVCQLCHPSPVHAAAHLSLETLTAFVLPFGLMLGCYSVTLARLRGARWGSGRHGARVGRLVSAIVLAFGLLWAPYHAVNLLQAVAALAPPEGALAKLGGAGQAARAGTTALAFFSSSVNPVLYVFTAGDLLPRAGPRFLTRLFEGSGEARGGGRSREGTMELRTTPQLKVVGQGRGNGDPGGGMEKDGPEWDL

SEQ ID NO: 3 - DNA, 1170 bp, Homo sapiens; identical to SEQ ID NO: 1
  CDS (94)..(1170)

SEQ ID NO: 4 - protein, 358 aa, Homo sapiens
MSVCYRPPGNETLLSWKTSRATGTAFLLLAALLGLPGNGFVVWSLAGWRPARGRPLAATLVLHLALADGAVLLLTPLFVAFLTRQAWPLGQAGCKAVYYVCALSMYASVLLTGLLSLQRCLAVTRPFLAPRLRSPALARRLLLAVWLAALLLAVPAAVYRHLWRDRVCQLCHPSPVHAAAHLSLETLTAFVLPFGLMLGCYSVTLARLRGARWGSGRHGARVGRLVSAIVLAFGLLWAPYHAVNLLQAVAALAPPEGALAKLGGAGQAARAGTTALAFFSSSVNPVLYVFTAGDLLPRAGPRFLTRLFEGSGEARGGGRSREGTMELRTTPQLKVVGQGRGNGDPGGGMEKDGPEWDL

SEQ ID NO: 5 - DNA, 1060 bp, Homo sapiens
  CDS (1)..(1059)
atgaacactacatcttctgcagcacccccctcactaggtgtagagttcatctctctgctggctatcatcctgctgtcagtggcgctggctgtggggcttcccggcaacagctttgtggtgtggagtatcctgaaaaggatgcagaagcgctctgtcactgccctgatggtgctgaacctggccctggccgacctggccgtattgctcactgctccctttttccttcacttcctggcccaaggcacctggagttttggactggctggttgccgcctgtgtcactatgtctgcggagtcagcatgtacgccagcgtcctgcttatcacggccatgagtctagaccgctcactggcggtggcccgcccctttgtgtcccagaagctacgcaccaaggcgatggcccggcgggtgctggcaggcatctgggtgttgtcctttctgctggccacacccgtcctcgcgtaccgcacagtagtgccctggaaaacgaacatgagcctgtgcttcccgcggtaccccagcgaagggcaccgggccttccatctaatcttcgaggctgtcacgggcttcctgctgcccttcctggctgtggtggccagctactcggacatagggcgtcggctacaggcccggcgcttccgccgcagccgccgcaccggccgcctggtggtgctcatcatcctgaccttcgccgccttctggctgccctaccacgtggtgaacctggctgaggcgggccgcgcgctggccggccaggccgccgggttagggctcgtggggaagcggctgagcctggcccgcaacgtgctcatcgcactcgccttcctgagcagcagcgtgaaccccgtgctgtacgcgtgcgccggcggcggcctgctgcgctcggcgggcgtgggcttcgtcgccaagctgctggagggcacgggttccgaggcgtccagcacgcgccgcgggggcagcctgggccagaccgctaggagcggccccgccgctctggagcccggcccttccgagagcctcactgcctccagccctctcaagttaaacgaactgaactagg

SEQ ID NO: 6 - protein, 352 aa, Homo sapiens
MNTTSSAAPPSLGVEFISLLAIILLSVALAVGLPGNSFVVWSILKRMQKRSVTALMVLNLALADLAVLLTAPFFLHFLAQGTWSFGLAGCRLCHYVCGVSMYASVLLITAMSLDRSLAVARPFVSQKLRTKAMARRVLAGIWVLSFLLATPVLAYRTVVPWKTNMSLCFPRYPSEGHRAFHLIFEAVTGFLLPFLAVVASYSDIGRRLQARRFRRSRRTGRLVVLIILTFAAFWLPYHVVNLAEAGRALAGQAAGLGLVGKRLSLARNVLIALAFLSSSVNPVLYACAGGGLLRSAGVGFVAKLLEGTGSEASSTRRGGSLGQTARSGPAALEPGPSESLTASSPLKLNELN

SEQ ID NO: 7 - DNA, 1134 bp, homo sapiens
  CDS (1)..(1134)
atggcgtcaggaaacccttggtcctctactctcatgcgtgtgtccgccctcactctccaggtcctcccgacggccatgaacactacatcttctgcagcacccccctcactaggtgtagagttcatctctctgctggctatcatcctgctgtcagtggcgctggctgtggggcttcccggcaacagctttgtggtgtggagtatcctgaaaaggatgcagaagcgctctgtcactgccctgatggtgctgaacctggccctggccgacctggccgtattgctcactgctccctttttccttcacttcctggcccaaggcacctggagttttggactggctggttgccgcctgtgtcactatgtctgcggagtcagcatgtacgccagcgtcctgcttatcacggccatgagtctagaccgctcactggcggtggcccgcccctttgtgtcccagaagctacgcaccaaggcgatggcccggcgggtgctggcaggcatctgggtgttgtcctttctgctggccacacccgtcctcgcgtaccgcacagtagtgccctggaaaacgaacatgagcctgtgcttcccgcggtaccccagcgaagggcaccgggccttccatctaatcttcgaggctgtcacgggcttcctgctgcccttcctggctgtggtggccagctactcggacatagggcgtcggctacaggcccggcgcttccgccgcagccgccgcaccggccgcctggtggtgctcatcatcctgaccttcgccgccttctggctgccctaccacgtggtgaacctggctgaggcgggccgcgcgctggccggccaggccgccgggttagggctcgtggggaagcggctgagcctggcccgcaacgtgctcatcgcactcgccttcctgagcagcagcgtgaaccccgtgctgtacgcgtgcgccggcggcggcctgctgcgctcggcgggcgtgggcttcgtcgccaagctgctggagggcacgggctccgaggcgtccagcacgcgccgcgggggcagcctgggccagaccgctaggagcggccccgccgctctggagcccggcccttccgagagcctcactgcctccagccctctcaagttaaacgaactgaactag

SEQ ID NO: 8 - protein, 377 aa, homo sapiens
MASGNPWSSTLMRVSALTLQVLPTAMNTTSSAAPPSLGVEFISLLAIILLSVALAVGLPGNSFVVWSILKRMQKRSVTALMVLNLALADLAVLLTAPFFLHFLAQGTWSFGLAGCRLCHYVCGVSMYASVLLITAMSLDRSLAVARPFVSQKLRTKAMARRVLAGIWVLSFLLATPVLAYRTVVPWKTNMSLCFPRYPSEGHRAFHLIFEAVTGFLLPFLAVVASYSDIGRRLQARRFRRSRRTGRLVVLIILTFAAFWLPYHVVNLAEAGRALAGQAAGLGLVGKRLSLARNVLIALAFLSSSVNPVLYACAGGGLLRSAGVGFVAKLLEGTGSEASSTRRGGSLGQTARSGPAALEPGPSESLTASSPLKLNELN